(19) United States
(12) Patent Application Publication
(10) Pub. No.: US 2024/0188945 A1
(43) Pub. Date: Jun. 13, 2024

(54) VASCULAR INTERVENTIONAL SURGERY DEVICE, AND DRUM ASSEMBLY FOR VASCULAR INTERVENTIONAL SURGERY DEVICE

(71) Applicant: PERAZAH INC., Ansan-si (KR)

(72) Inventors: Hwan Taek Ryu, Ansan-si (KR); Jong Tae Seo, Ansan-si (KR)

(21) Appl. No.: 18/444,441

(22) Filed: Feb. 16, 2024

Related U.S. Application Data
(63) Continuation of application No. PCT/KR2022/015642, filed on Oct. 14, 2022.

(30) Foreign Application Priority Data
Oct. 15, 2021 (KR) 10-2021-0137714
Feb. 11, 2022 (KR) 10-2022-0018176

(51) Int. Cl. A61B 17/00; A61M 25/01

(52) U.S. Cl. CPC A61B 17/00234; A61B 2017/00318; A61B 2017/00477; A61B 2017/00778; A61M 25/0136

(57) ABSTRACT

According to a disclosed embodiment, a vascular interventional surgery device includes: a fixed frame; a steering assembly coupled to the fixed frame so as to be rotatable about a first axis, and including a first introduction/discharge port aligned on the first axis; and a drum assembly coupled to the steering assembly so as to be rotatable about a second axis extending in a direction that is transverse to the first axis, including a rotation unit configured to allow a flexible wire-type or tubular surgical tool that is insertable into a vessel to be wound in a circumferential direction about the second axis, and configured to guide the surgical tool such that the surgical tool is withdrawn outward or inserted inward through the first introduction/discharge port when the rotation unit rotates about the second axis.

### CROSS-REFERENCE TO RELATED APPLICATIONS

This application is a Continuation of Application No. PCT/KR2022/015642, filed on Oct. 14, 2022, which in turn claims the benefit of Korean Patent Applications No. 10-2021-0137714, filed on Oct. 15, 2021, and No. 10-2022-0018176, filed on Feb. 11, 2022. The entire disclosures of all these applications are hereby incorporated by reference.

### TECHNICAL FIELD

The present disclosure relates to a vascular interventional surgery device. More particularly, the present disclosure relates to a vascular interventional surgery device capable of controlling a flexible wire-type or tubular surgical tool that is insertable into a vessel.

### BACKGROUND ART

Vascular interventional surgery refers to a minimally invasive surgery aimed at treating vascular diseases or cancer, in which the treatment is mostly performed by percutaneously inserting a thin catheter having a diameter of several millimeters or less to a lesion site through a vessel under X-ray fluoroscopy to allow the catheter to reach a target organ. Representative treatments adopting the vascular interventional surgery currently being performed around the world, including Korea, include trans-arterial chemoembolization (TACE) for liver cancer, percutaneous angioplasty, endovascular stent graft placement for aortic diseases, and the like.

Most vessels are divided into several branches, or have curved shapes. Therefore, in order to prevent damage to vessels, surgical tools having multiple levels of diameters, which are referred as a co-axial system of a catheter and a guide wire, may overlap each other so as to be used in the vascular interventional surgery. In this case, since the vessel has a branching point in which the vessel is divided into several branches or a curved region, an operator has to manually steer and insert the catheter and the guide wire precisely in a direction of the vessel.

A conventional device for inserting, extracting, or steering a surgical tool has a complex structure, so that it may be inconvenient to replace the surgical tool that is used once or clean equipment that is contaminated together with the surgical tool.

### DISCLOSURE

### Technical Problem

An object of the present disclosure is to provide a vascular interventional surgery device having a simple structure and capable of facilitating replacement of a surgical tool.

### Technical Solution

According to one embodiment of the present disclosure, a vascular interventional surgery device includes: a fixed frame; a steering assembly coupled to the fixed frame so as to be rotatable about a first axis, and including a first introduction/discharge port aligned on the first axis; and a drum assembly coupled to the steering assembly so as to be rotatable about a second axis extending in a direction that is transverse to the first axis, including a rotation unit configured to allow a flexible wire-type or tubular surgical tool that is insertable into a vessel to be wound in a circumferential direction about the second axis, and configured to guide the surgical tool such that the surgical tool is withdrawn outward or inserted inward through the first introduction/discharge port when the rotation unit rotates about the second axis.

According to one embodiment of the present disclosure, a drum assembly for a vascular interventional surgery device includes: a guide unit including an introduction/discharge port aligned on a first axis; and a rotation unit coupled to the guide unit so as to be rotatable about a second axis extending in a direction that is transverse to the first axis, and configured to allow a flexible wire-type or tubular surgical tool that is insertable into a vessel to be wound in a circumferential direction about the second axis, wherein the guide unit is configured to guide the surgical tool such that the surgical tool advances outward or retracts inward through the introduction/discharge port when the rotation unit rotates about the second axis with respect to the guide unit.

### Advantageous Effects

According to embodiments of the present disclosure, a vascular interventional surgery device having a simple structure and capable of facilitating replacement of a surgical tool can be provided.

### DESCRIPTION OF DRAWINGS

FIG. 1 is a perspective view showing a vascular interventional surgery device according to one embodiment.

FIG. 2 is an exploded perspective view showing the vascular interventional surgery device according to one embodiment.

FIG. 3 is an exploded perspective view showing a drum assembly according to one embodiment when viewed from a top.

FIG. 4 is an exploded perspective view showing the drum assembly according to one embodiment when viewed from a bottom.

FIG. 5a shows assembly of a first fixing member and a rotation unit.

FIG. 5b shows a surgical tool inserted into the first fixing member.

FIG. 5c shows assembly of a second fixing member.

FIG. 5d shows a state in which the surgical tool is further inserted into a second reception part.

FIG. 5e, and FIG. 5f show a movement direction of the surgical tool according to rotation of the rotation unit.

FIG. 5g is a perspective view showing an operation of the first fixing member that fixes the surgical tool, and an enlarged sectional view showing a portion thereof.

FIG. 6 shows an operation of a locking member of the drum assembly according to one embodiment.

FIG. 7 is a sectional view showing the drum assembly of FIG. 6 taken along a line I-I′ and a line II-II′.

FIG. 8 is an exploded perspective view showing a steering assembly according to one embodiment when viewed from the top.

FIG. 9 is an exploded perspective view showing the steering assembly according to one embodiment when viewed from the bottom.

FIG. 10 shows a translational interworking shaft and a docking shaft coupled thereto.

FIG. 11a shows draping of a portion of the steering assembly.

FIG. 11b, FIG. 11c, FIG. 11d and FIG. 11e show a process of coupling a fastening member to a guide pipe.

FIG. 12 is a top view showing the steering assembly on which the drum assembly is mounted according to one embodiment.

FIG. 13 is an exploded sectional view showing the steering assembly and the drum assembly.

FIG. 14 shows an operation of engaging the docking shaft with the drum assembly.

FIG. 15 shows a process of fixing the drum assembly to the steering assembly.

FIG. 16 is an exploded perspective view showing a base according to one embodiment.

FIG. 17 shows a driving unit of the vascular interventional surgery device according to one embodiment.

FIG. 18 is a sectional view showing the driving unit of the vascular interventional surgery device.

### MODE FOR INVENTION

Embodiments of the present disclosure are illustrated for the purpose of describing the technical idea of the present disclosure. The scope of rights according to the present disclosure is not limited to the embodiments presented below or specific descriptions of the embodiments.

Unless defined otherwise, all technical terms and scientific terms used in the present disclosure have meanings commonly understood by a person having ordinary skill in the art to which the present disclosure pertains. All terms used in the present disclosure are selected for the purpose of more clearly describing the present disclosure, but not selected to limit the scope of rights according to the present disclosure.

Expressions such as “comprising”, “including”, and “having” used in the present disclosure should be understood as open-ended terms that imply possibility of including other embodiments unless stated otherwise in a phrase or a sentence including the expression.

An expression in a singular form used in the present disclosure may include a meaning of a plural form unless stated otherwise, and this also applies to an expression in a singular form used in the claims.

Expressions such as “first” and “second” used in the present disclosure are used to distinguish a plurality of elements from each other, but not to limit an order or importance of the elements.

Dimensions and numerical values described in the present disclosure are not limited to only the described dimensions and numerical values. Unless specified otherwise, the dimensions and the numerical values may be understood to mean described values and equivalent ranges encompassing the described values. For example, a dimension “** mm” described in the present disclosure may be understood to include “about ** mm”.

Hereinafter, the embodiments of the present disclosure will be described with reference to the accompanying drawings. Throughout the accompanying drawings, identical or corresponding elements are given the same reference numerals. In addition, while describing the following embodiments, redundant descriptions of the identical or corresponding elements may be omitted. However, even when the descriptions of the elements are omitted, the elements are not intended to be excluded from any embodiment.

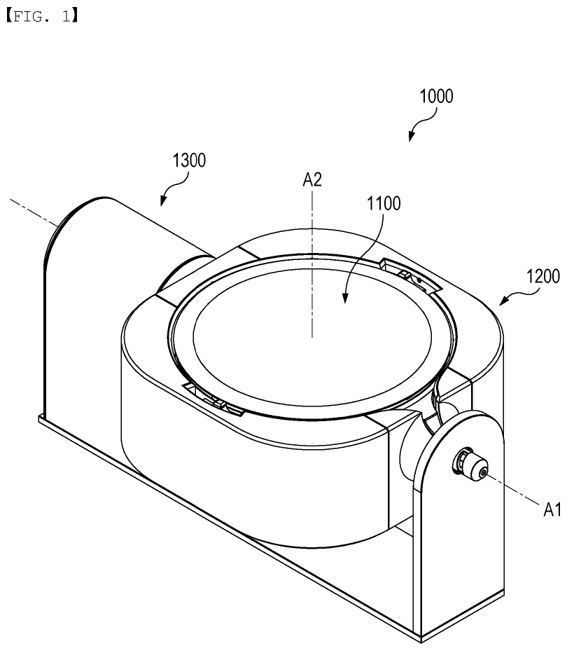

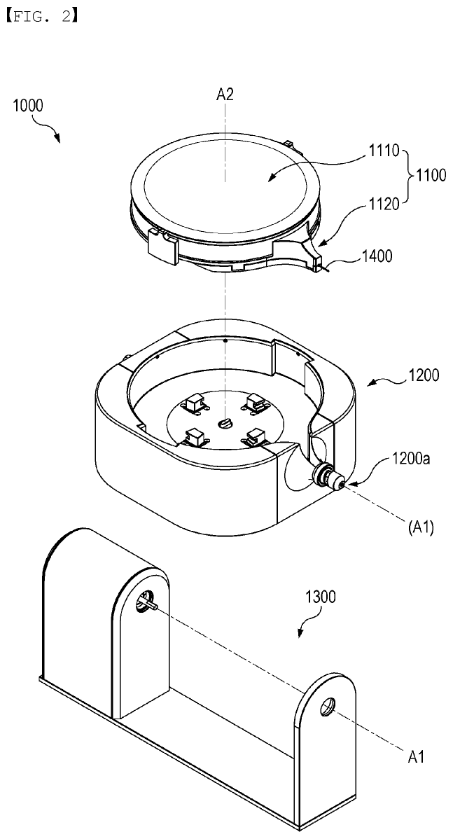

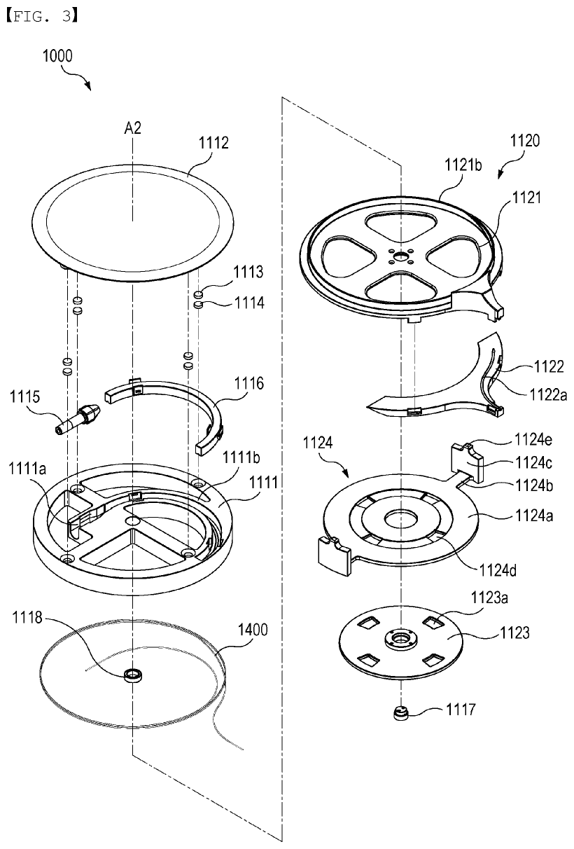

FIG. 1 is a perspective view showing a vascular interventional surgery device 1000 according to one embodiment. FIG. 2 is an exploded perspective view showing the vascular interventional surgery device 1000 according to one embodiment.

Referring to FIGS. 1 and 2, a vascular interventional surgery device 1000 may include a drum assembly 1100, a steering assembly 1200, and a base 1300.

According to one embodiment, the steering assembly 1200 may be rotatably coupled to the base 1300. The steering assembly 1200 may rotate about a first axis A1 with respect to the base 1300. The “first axis A1” used in the present disclosure refers to a virtual axis as shown in FIG. 1. The steering assembly 1200 may include a first introduction/discharge port 1200a aligned on the first axis A1.

A surgical tool 1400 may be inserted into the drum assembly 1100, and a portion of the surgical tool 1400 may be withdrawn out of the vascular interventional surgery device 1000 from the drum assembly 1100.

The surgical tool 1400 may be a member that is insertable into a vessel. The surgical tool 1400 may be a flexible wire-type or tubular member. For example, the surgical tool 1400 may be a catheter, a guide wire inserted into the catheter, a micro-catheter that is insertable into the catheter, or a micro-guide wire that is insertable into the micro-catheter.

The surgical tool 1400 withdrawn out of the drum assembly 1100 may escape through a first introduction/discharge port 1200a formed in the steering assembly 1200.

The drum assembly 1100 may be configured to adjust a length of the surgical tool 1400 withdrawn out of the vascular interventional surgery device 1000. The drum assembly 1100 may include a guide unit 1120, and a rotation unit 1110 rotatably coupled to the guide unit 1120. As the rotation unit 1110 rotates about a second axis A2, the surgical tool 1400 may advance or retreat. The “second axis A2” used in the present disclosure refers to a virtual axis as shown in FIG. 1.

According to one embodiment, the drum assembly 1100 may be configured to move the surgical tool 1400 forward or rearward in a direction of the first axis A1. The surgical tool 1400 may be wound inside the drum assembly 1100 about the second axis A2. As the rotation unit 1110 rotates about the second axis A2, the surgical tool 1400 may further come out of the drum assembly 1100 or further get into the drum assembly 1100.

The drum assembly 1100 may be coupled to the steering assembly 1200. The drum assembly 1100 may be detachably coupled to the steering assembly 1200.

The steering assembly 1200 may be configured to rotate the drum assembly 1100 about the first axis A1. Accordingly, the steering assembly 1200 may rotate the surgical tool 1400 withdrawn through the first introduction/discharge port 1200a.

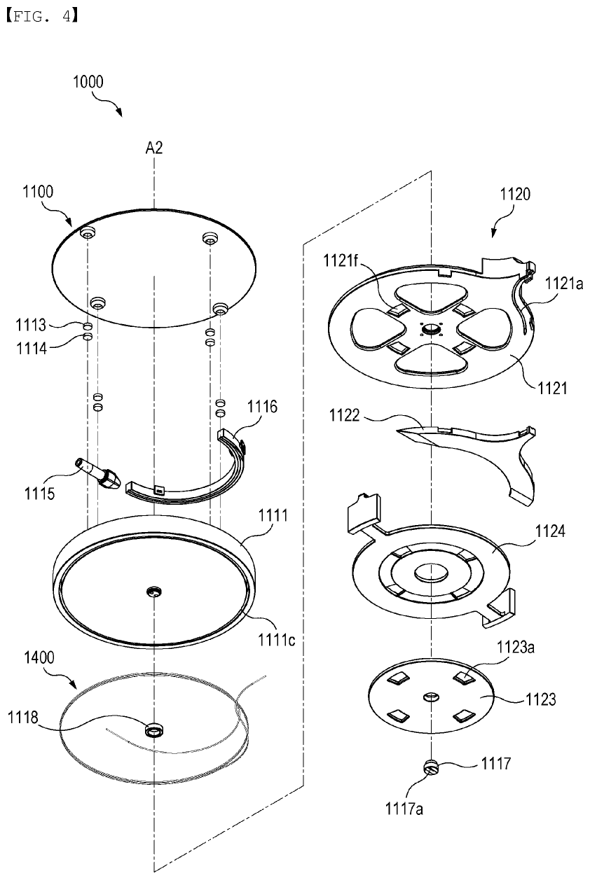

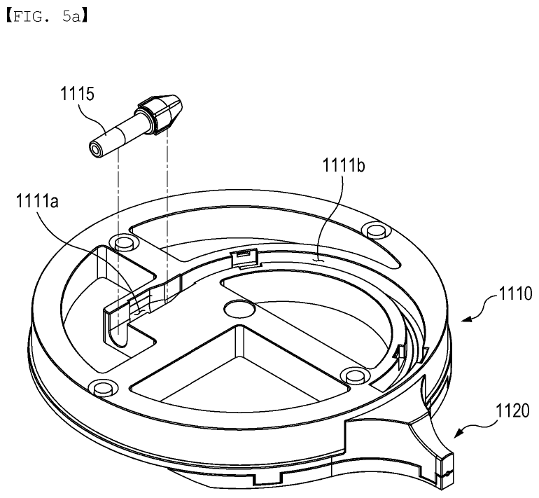

FIG. 3 is an exploded perspective view showing a drum assembly 1100 according to one embodiment when viewed from a top. FIG. 4 is an exploded perspective view showing the drum assembly 1100 according to one embodiment when viewed from a bottom. Hereinafter, the drum assembly 1100 will be described with reference to FIGS. 3 and 4.

According to one embodiment, the drum assembly 1100 may include a guide unit 1120 and a rotation unit 1110. The rotation unit 1110 may be rotatably coupled to the guide unit 1120. The rotation unit 1110 may rotate about the second axis A2 with respect to the guide unit 1120.

The rotation unit 1110 may be configured to receive the surgical tool 1400. For example, the rotation unit 1110 may include a first reception part 1111a formed in an upper portion of the rotation unit 1110, a second reception part 1111b, and a third reception part 1111c formed in a lower portion of the rotation unit 1110. A portion of the surgical tool 1400 may be disposed in the first to third reception parts 1111a, 1111b, and 1111c.

The guide unit 1120 may include a first part 1121, and a second part 1122 coupled to the first part 1121. According to another embodiment, the first part 1121 and the second part 1122 may be formed integrally with each other.

The drum assembly 1100 may include fixing members 1115 and 1116 configured to fix the surgical tool 1400 to the rotation unit 1110. For example, the drum assembly 1100 may include a first fixing member 1115 inserted into the first reception part 1111a, and a second fixing member 1116 inserted into the second reception part 1111b.

The drum assembly 1100 may include a cover 1112 coupled to the upper portion of the rotation unit 1110. The cover 1112 may be configured to cover the upper portion of the rotation unit 1110. The cover 1112 may protect elements (e.g., the surgical tool 1400) disposed in the upper portion of the rotation unit 1110. The cover 1112 may form an exterior of the vascular interventional surgery device 1000.

The cover 1112 may be detachably coupled to the rotation unit 1110. According to one embodiment, the cover 1112 and the rotation unit 1110 may be coupled to each other through a magnetic force. For example, a first magnetic member 1113 and a second magnetic member 1114, which generate magnetic attraction between each other, may be coupled to the cover 1112 and the rotation unit 1110, respectively. Referring to FIGS. 3 and 4, the first magnetic member 1113 (or the second magnetic member 1114) may be disposed at each of four positions arranged in a circumferential direction on an outer periphery of the cover 1112 (or the rotation unit 1110), but the above configuration is provided merely for illustrative purposes, and a plurality of magnetic members may be arranged in a different number or a different arrangement as compared with the shown embodiment.

The guide unit 1120 may be configured to guide the surgical tool 1400 when the surgical tool 1400 is withdrawn out of the drum assembly 1100 or inserted into the drum assembly 1100. The guide unit 1120 may include a passage configured to guide the surgical tool 1400.

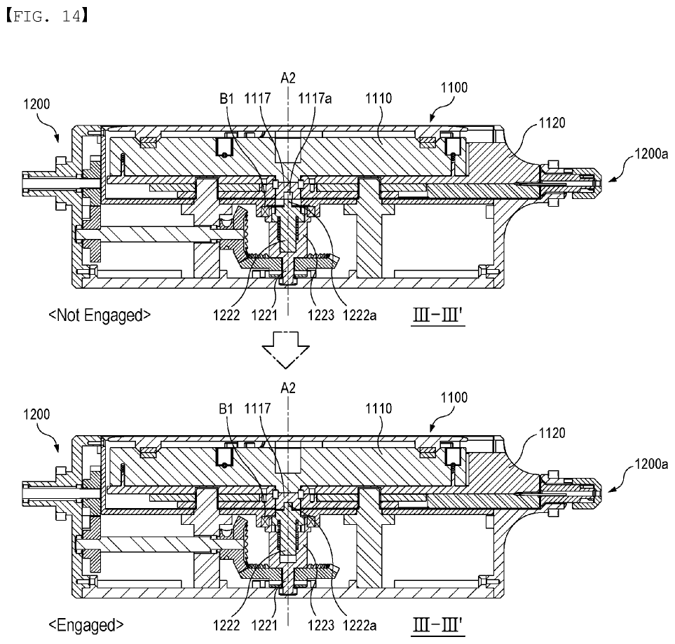

The vascular interventional surgery device 1000 may include a mechanism configured to fix the drum assembly 1100 to the steering assembly 1200. The drum assembly 1100 may include a locking member 1124. The locking member 1124 may be selectively coupled to a partial element of the steering assembly 1200 after the drum assembly 1100 is inserted into the steering assembly 1200, so that the drum assembly 1100 may be fixed to the steering assembly 1200. A coupling mechanism between the drum assembly 1100 and the steering assembly 1200 using the locking member 1124 will be described in detail with reference to FIG. 14.

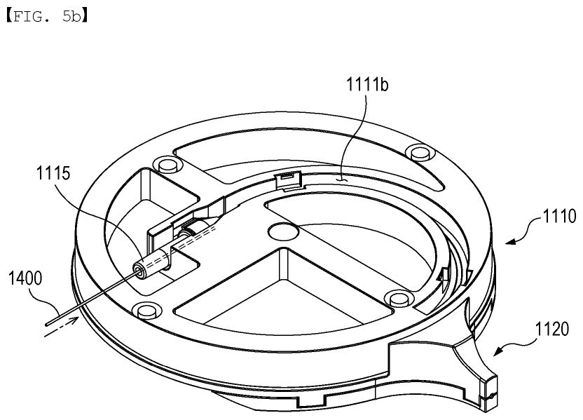

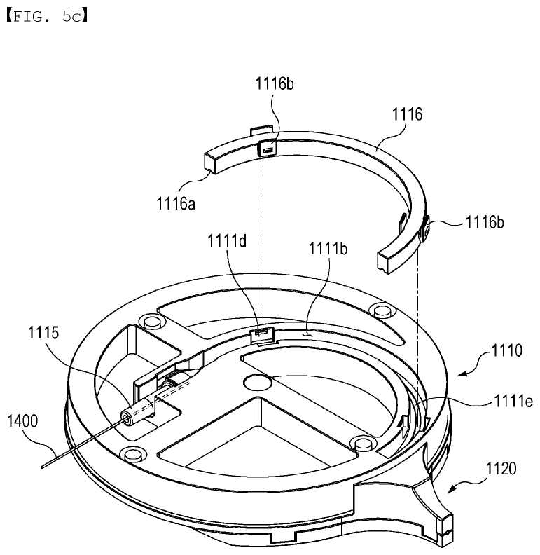

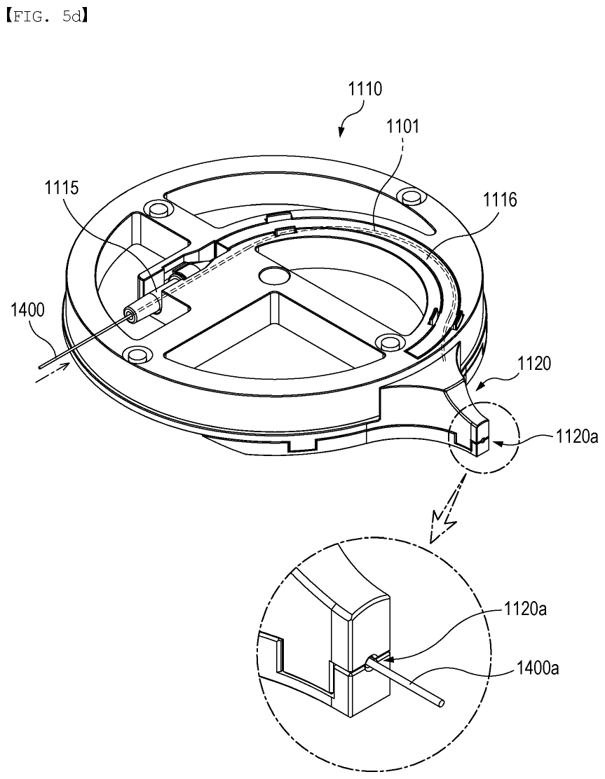

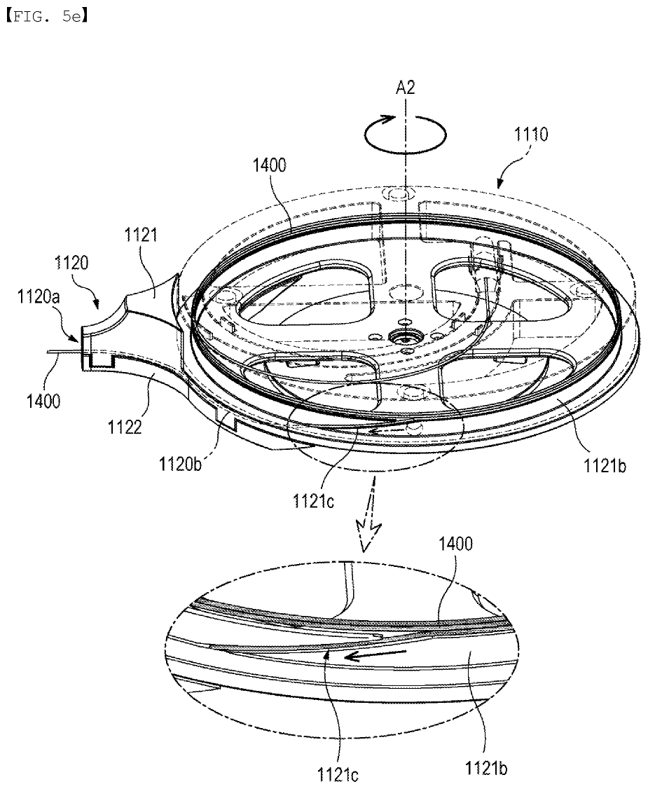

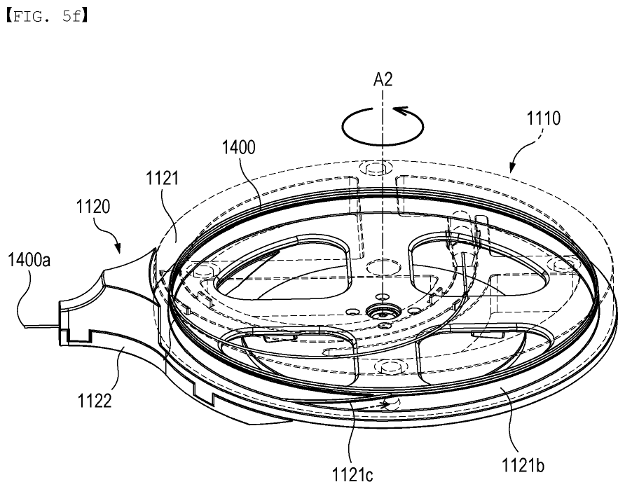

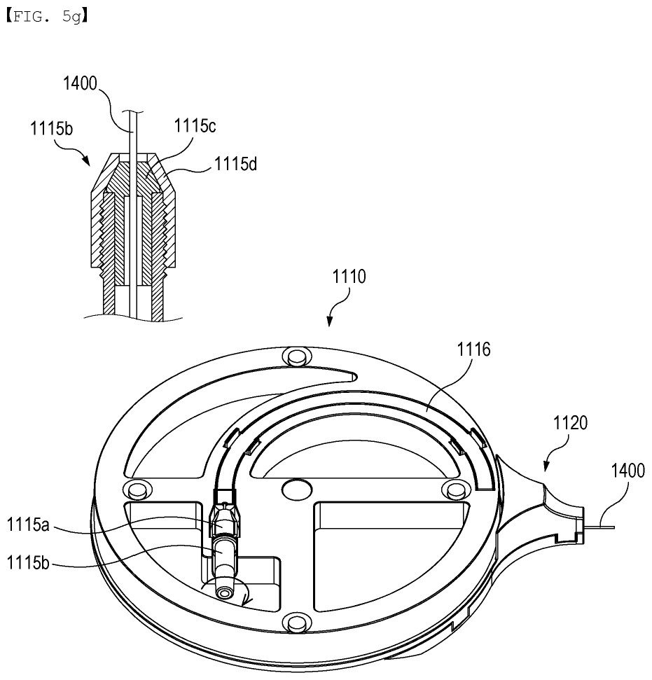

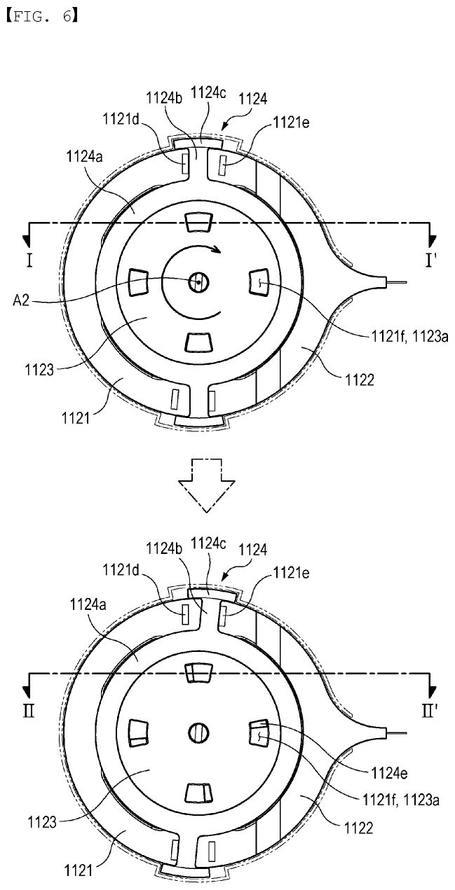

FIG. 5a shows assembly of a first fixing member 1115 and a rotation unit. FIG. 5b shows a surgical tool 1400 inserted into the first fixing member 1115. FIG. 5c shows assembly of a second fixing member 1116. FIG. 5d shows a state in which the surgical tool 1400 is further inserted into a second reception part 1111b. FIGS. 5e and 5f show a movement direction of the surgical tool 1400 according to rotation of the rotation unit 1110. FIG. 5g is a perspective view showing an operation of the first fixing member 1115 that fixes the surgical tool 1400, and an enlarged sectional view showing a portion thereof.

Referring to FIGS. 5a to 5f, after the first fixing member 1115 is assembled with the first reception part 1111a formed in the upper portion of the rotation unit 1110, the surgical tool 1400 may be inserted into the first fixing member 1115. Alternatively, the surgical tool 1400 may be first inserted into the first fixing member 1115, and the first fixing member 1115 may be assembled with the first reception part 1111a.

When the surgical tool 1400 is pushed further into the first fixing member 1115, the surgical tool 1400 may escape the first fixing member 1115 so as to be inserted into a first passage 1101 formed between the second reception part 1111b and the second fixing member 1116. According to one embodiment, the first passage 1101 may be defined by a first guide groove 1116a formed on a bottom surface of the second fixing member 1116 and/or a second guide groove 1111e formed on a floor surface of the second reception part 1111b. The first guide groove 1116a and the second guide groove 1111e may extend in a semicircular shape, so that the surgical tool 1400 may also be bent in a semicircular shape and arranged in the second reception part 1111b.

A coupling structure 1116b of the second fixing member 1116 may be coupled to a corresponding structure 1111d of the rotation unit 1110. The coupling structure 1116b and the corresponding structure 1111d shown in the drawings are provided merely in an exemplary form, and according to other embodiments, the second fixing member 1116 and the rotation unit 1110 may be coupled to each other in various ways.

The rotation unit 1110 may be configured to allow the surgical tool 1400 to be wound in a circumferential direction about the second axis A2. The rotation unit 1110 may include a third reception part 1111c formed in the lower portion of the rotation unit 1110 and configured to receive the surgical tool 1400. For example, the third reception part 1111c may be provided in the form of a groove extending in the circumferential direction about the second axis A2 in the lower portion of the rotation unit 1110, and the surgical tool 1400 may be wound in the third reception part 1111c.

The third reception part 1111c may communicate with the second reception part 1111b. Referring to FIGS. 4 and 5e, as the surgical tool 1400 is further introduced into the rotation unit 1110, the surgical tool 1400 may be introduced into the third reception part 1111c formed in the lower portion of the rotation unit 1110.

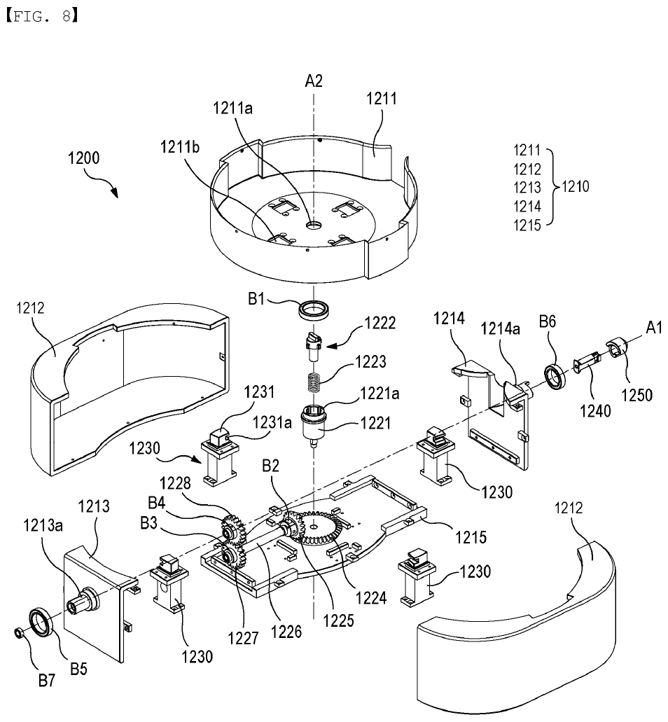

Referring to FIG. 5e, the guide unit 1120 may include a rib 1121b received in the third reception part 1111c of the rotation unit 1110. The rib 1121b may be disposed in an upper portion of the guide unit 1120 to extend in the circumferential direction about the second axis A2. For example, the rib 1121b may be provided in the form of a wall extending in the circumferential direction about the second axis A2. As the rotation unit 1110 is coupled to the guide unit 1120, the rib 1121b may be received in the third reception part 1111c. Referring to an upper drawing of FIG. 7, a gap may be formed between an upper end of the rib 1121b and a floor surface of the third reception part 1111c, and the surgical tool 1400 may be located in the gap.

Referring to FIG. 5e, a portion of the surgical tool 1400 located in the third reception part 1111c may be introduced into a second passage 1121c formed in the rib 1121b as the rotation unit 1110 rotates clockwise about the second axis A2. The second passage 1121c may extend 10 in a direction between a direction that is parallel to the second axis A2 and the circumferential direction of the second axis A2. For example, the second passage 1121c may be partially defined by an inclined surface formed on the rib 1121b. For another example, the second passage 1121c may be provided in the form of an inclined slit formed in the rib 1121b.

A portion of the surgical tool 1400 located in the second passage 1121c may get into a third passage 1120b communicating with the second passage 1121c as the rotation unit 1110 rotates clockwise about the second axis A2. The third passage 1120b may be configured to guide the surgical tool 1400 to a second introduction/discharge port 1120a of the drum assembly 1100. The third passage 1120b may be formed inside the guide unit 1120. For example, referring to FIGS. 3 and 4 together, the third passage 1120b may be defined by coupling the first part 1121 and the second part 1122 to each other. A third guide groove 1121a may be disposed on a bottom surface of the first part 1121, and a fourth guide groove 1122a corresponding to the third guide groove 1121a may be disposed on a top surface of the second part 1122. As the first part 1121 and the second part 1122 are coupled to each other, the third guide groove 1121a and the fourth guide groove 1122a may form the third passage 1120b, and the surgical tool 1400 may move along the third passage 1120b.

Referring to FIG. 5f, as the rotation unit 1110 rotates counterclockwise about the second axis A2 with respect to the guide unit 1120, a portion of the surgical tool 1400 may be wound back into the third reception part 1111c from the second passage 1121c, so that a front end portion 1400a of the surgical tool 1400 may retract.

When the surgical tool 1400 is initially inserted into the drum assembly 1100, the surgical tool 1400 may be directly introduced into the second passage 1121c without being wound in the third reception part 1111c. In this case, the surgical tool 1400 may escape out of the second introduction/discharge port 1120a so as to be elongated. Referring to FIG. 5f, when the rotation unit 1110 rotates counterclockwise about the second axis A2, the surgical tool 1400 elongated and extending out of the drum assembly 1100 may be pulled and wound inside the third reception part 1111c.

According to one embodiment, as the rotation unit 1110 rotates about the second axis A2, the surgical tool 1400 may be withdrawn or inserted through the second introduction/discharge port 1120a aligned on the first axis A1. As the rotation unit 1110 rotates about the second axis A2, the surgical tool 1400 may advance or retreat along the first axis A1 in a region that is adjacent to the second introduction/discharge port 1120a aligned on the first axis A1.

Referring to FIG. 1 together, the first axis A1 on which the second introduction/discharge port 1120a is aligned refers to a rotation axis of the steering assembly 1200 with respect to the base 1300. As the rotation unit 1110 rotates about the second axis A2, the surgical tool 1400 may advance or retreat, and as the steering assembly 1200 to which the rotation unit 1110 is coupled rotates about the first axis A1, the surgical tool 1400 may rotate. The second axis A2 may extend in a direction that is transverse to the first axis A1. For example, the first axis A1 and the second axis A2 may be perpendicular or substantially perpendicular to each other.

Referring to FIG. 5g, the first fixing member 1115 may be provided in the form of a torque device. Referring to the enlarged sectional view of FIG. 5g, the first fixing member 1115 may include an operation part 1115a gripped and operated by an operator. The first fixing member 1115 may include a head part 1115b configured to grip a base end portion of the surgical tool 1400 on a front end side of the operation part 1115a. The head part 1115b may include a chuck claw 1115c. The head part 1115b may include a screw cap 1115d configured to clamp the chuck claw 1115c. A female screw thread coupled to a male screw thread formed at a front end of the operation part 1115a may be formed on an inner peripheral surface of the screw cap 1115d. When the screw cap 1115d is fastened to the front end of the operation part 1115a by rotation the operation part 1115a, the screw cap 1115d may press the chuck claw 1115c from a periphery of the chuck claw 1115c to firmly grip the surgical tool 1400. Meanwhile, the form of the torque device may vary, and the embodiment of the present disclosure is not limited to the example shown in FIG. 5g.

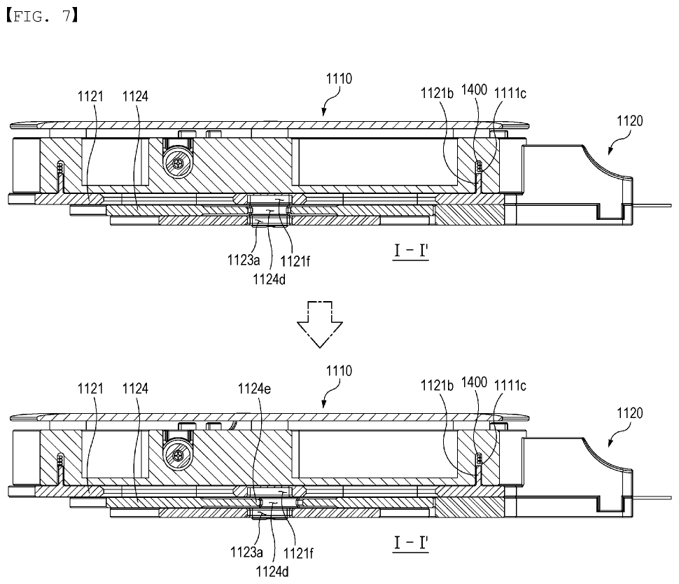

FIG. 6 shows an operation of a locking member 1124 of the drum assembly 1100 according to one embodiment. FIG. 7 is a sectional view showing the drum assembly 1100 of FIG. 6 taken along a line I-I′ and a line II-II′.

Referring to FIGS. 3 and 6, the locking member 1124 may include a rotation plate 1124a, an arm 1124b extending outward from the rotation plate 1124a, and a lever 1124c extending from the arm 1124b to an upper side of the drum assembly 1100. The rotation plate 1124a may be coupled to the guide unit 1120 so as to be rotatable about the second axis A2. The rotation unit 1110 may receive a rotational moment that is parallel to the second axis A2 through the arm 1124b and the lever 1124c. For example, a user may rotate the locking member 1124 by pushing the lever 1124c. The guide unit 1120 may include a third part 1123 disposed in a lower portion of the locking member 1124. The third part 1123 may be coupled to the second part 1122, and may prevent the locking member 1124 from being separated.

The guide unit 1120 may include stoppers 1121d and 1121e configured to limit a rotation range of the locking member 1124. Referring to FIG. 6, the stoppers 1121d and 1121e located on both sides of the arm 1124b may be formed in the second part 1122 of the guide unit 1120. For example, the stoppers 1121d and 1121e may be provided in a form that protrudes more than a periphery thereof. An upper drawing of FIG. 6 shows a state in which the locking member 1124 is rotated counterclockwise as much as possible, and a lower drawing of FIG. 6 shows a state in which the locking member 1124 is rotated clockwise as much as possible.

Referring to FIGS. 6 and 7, a recess 1121f may be formed in the second part 1122, and holes 1124d and 1123a having sizes corresponding to the recess 1121f of the second part 1122 may be formed in the locking member 1124 and the third part 1123, respectively. Since the third part 1123 is fixedly coupled to the second part 1122, an alignment of the third part 1123 and the second part 1122 may be maintained. However, as the locking member 1124 rotates with respect to the guide unit 1120, the recess 1121f of the second part 1122 and the hole 1124d of the locking member 1124 may be selectively aligned with each other.

Referring to the upper drawing of FIG. 6 and the I-I′ sectional view of FIG. 7 (the upper drawing of FIG. 7), the recess 1121f formed in the second part 1122, the hole 1124d formed in the locking member 1124, and the hole 1123a formed in the third part 1123 may all be aligned with each other. Referring to the lower drawing of FIG. 6 and the II-II′ sectional view (a lower drawing of FIG. 7), the recess 1121f and the hole 1124d of the locking member 1124 may be misaligned with each other, so that a portion of the locking member 1124 may overlap the recess 1121f or the hole 1123a of the third part 1123 when viewed in the direction that is parallel to the second axis A2. The operation of the locking member 1124 described above is a mechanism for fixing the drum assembly 1100 to the steering assembly 1200, which will be described below with reference to FIG. 14.

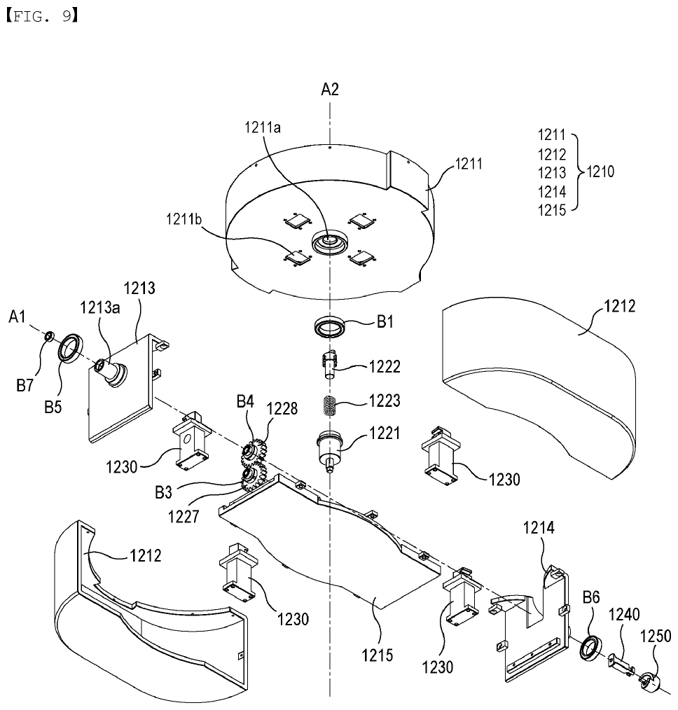

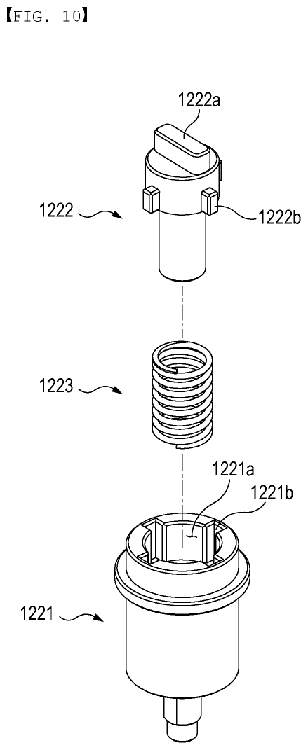

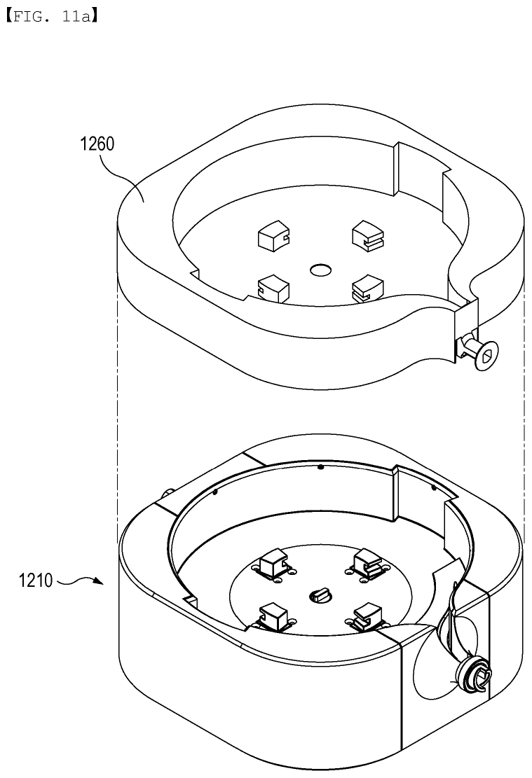

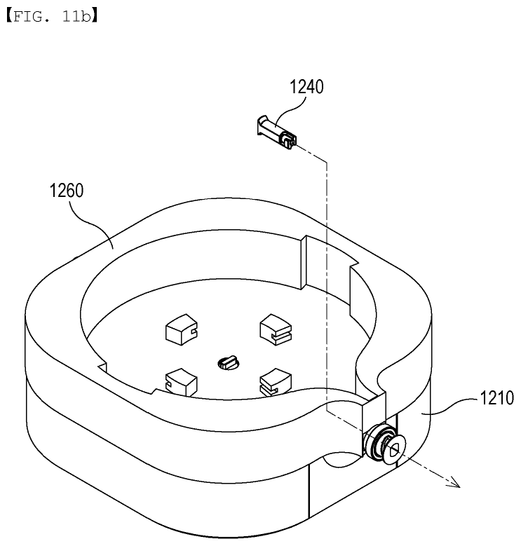

FIG. 8 is an exploded perspective view showing a steering assembly 1200 according to one embodiment when viewed from the top. FIG. 9 is an exploded perspective view showing the steering assembly 1200 according to one embodiment when viewed from the bottom. FIG. 10 shows a translational interworking shaft 1221 and a docking shaft 1222 coupled thereto.

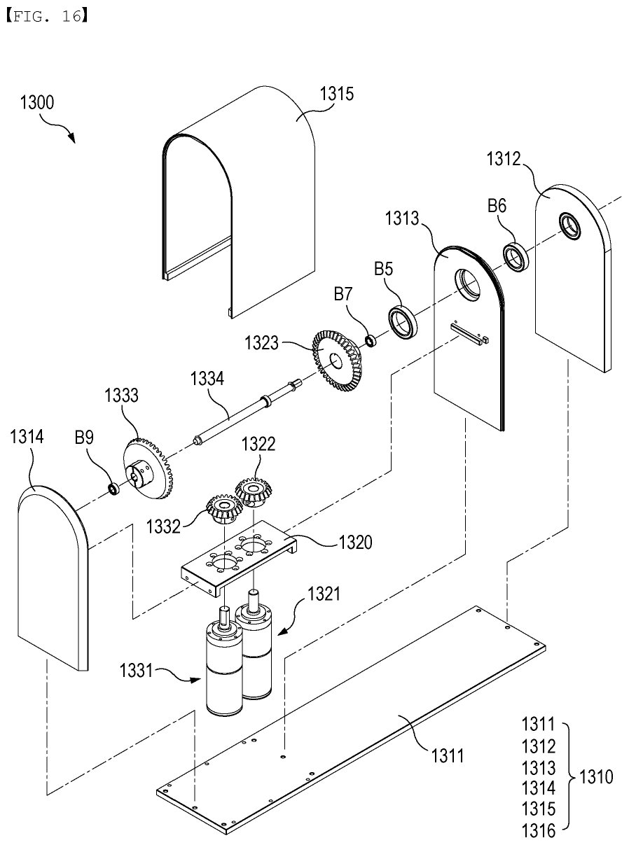

Referring to FIGS. 8 and 9, the steering assembly 1200 may include a housing 1210 configured to receive the drum assembly 1100. The housing 1210 may include an upper housing 1211, a lower housing 1215, a first side housing 1212, a second side housing 1213, and a third side housing 1214. The second side housing 1213 and the third side housing 1214 may include a steering driving shaft 1213a and a support shaft 1214a extending in the direction of the first axis A1, respectively. The steering driving shaft 1213a and the support shaft 1214a arranged on both sides of the housing 1210 in the direction of the first axis A1 may be rotatably coupled to a fixed frame (e.g., a first support frame 1312 and a second support frame 1313 of FIG. 16) to guide or support rotation of the steering assembly 1200.

The steering assembly 1200 may include a post 1230 engaged with the locking member 1124 of the drum assembly 1100 to allow the drum assembly 1100 to be fixed to the steering assembly 1200. A portion of the post 1230 may be located inside the housing 1210, and a portion 1231 of the post 1230 may protrude out of the upper housing 1211 through a hole 1211b formed in the upper housing 1211.

Referring to FIGS. 8 to 10, the steering assembly 1200 may include a translational interworking shaft 1221 and a docking shaft 1222. The docking shaft 1222 may be engaged with the rotation unit 1110 of the drum assembly 1100. For example, referring to FIG. 3 together, a first protrusion part 1222a formed on an upper portion of the docking shaft 1222 may be engaged with a connection part 1117 of the rotation unit 1110. When the translational interworking shaft 1221 rotates about the second axis A2 while the docking shaft 1222 is engaged with the rotation unit 1110, a torque of the translational interworking shaft 1221 may be transmitted to the connection part 1117 through the docking shaft 1222, and the rotation unit 1110 may rotate about the second axis A2.

The docking shaft 1222 may be retractably coupled to translational interworking shaft 1221. The translational interworking shaft 1221 may include a hollow 1221a formed inside the translational interworking shaft 1221 and extending along the second axis A2. A portion of the docking shaft 1222 may be received in the hollow 1221a.

According to one embodiment, the docking shaft 1222 may be configured to retreat or advance in a direction of the second axis A2 with respect to the translational interworking shaft 1221. A groove 1221b extending parallel to the second axis A2 may be formed inside the translational interworking shaft 1221. A second protrusion part 1222b formed at an outer periphery of the docking shaft 1222 may be inserted into the groove 1221b. As the second protrusion part 1222b is inserted into the groove 1221b, the docking shaft 1222 may be guided to move in a direction in which the groove 1221b extends (i.e., the direction of the second axis A2) with respect to the translational interworking shaft 1221. Since the second protrusion part 1222b is inserted into the groove 1221b, the docking shaft 1222 may not rotate relative to the translational interworking shaft 1221, and a rotational force transmitted to the translational interworking shaft 1221 may be transmitted to the rotation unit 1110 of the drum assembly 1100.

The docking shaft 1222 may elastically retract relative to the translational interworking shaft 1221. An elastic member 1223 configured to provide an elastic force to push the docking shaft 1222 when the docking shaft 1222 retracts may be provided. The elastic member may use one of various known schemes for exerting an elastic force. For example, the elastic member may include various types of members such as a compression spring, a tension spring, a torque spring, and an air spring. According to one embodiment, the elastic member 1223 may be interposed between the docking shaft 1222 and the translational interworking shaft 1221. For example, a coil spring may be inserted into the hollow 1221a of the translational interworking shaft 1221, and the docking shaft 1222 may be seated on the coil spring. When the docking shaft 1222 retracts into the hollow 1221a, the coil spring may be elastically compressed.

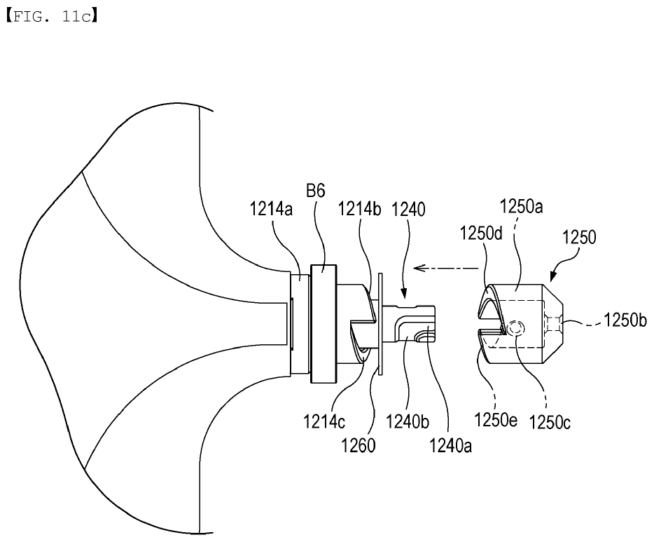

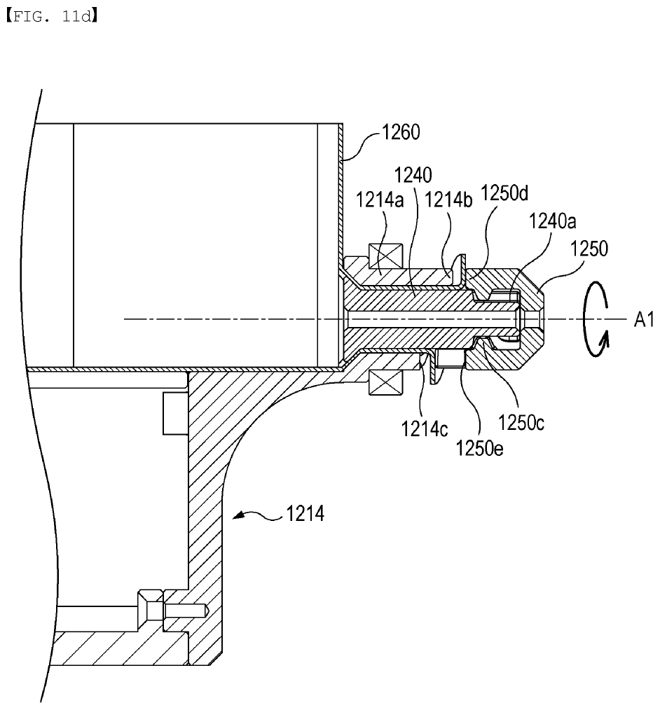

FIG. 11a shows draping of a portion of the steering assembly 1200. FIGS. 11b to 11e show a process of coupling a fastening member to a guide pipe.

Referring to FIG. 11a, a drap 1260 may be covered on an upper portion of the steering assembly 1200. According to one embodiment, the vascular interventional surgery device 1000 may be easily reused by covering the upper portion of the steering assembly 1200 with the drap 1260. The drap 1260 may prevent the steering assembly 1200 from being contaminated by substances put on the surgical tool 1400, and the drum assembly 1100 that is already contaminated may be easily separated from the surgical tool 1400. In other words, the user may easily reuse the vascular interventional surgery device 1000 by replacing only the drap 1260 and the drum assembly 1100.

Referring to FIGS. 11b to 11e, a guide pipe 1240 may be fitted to the steering assembly 1200 covered with the drap 1260, and may partially protrude out of a second steering driving shaft 1213a. A fastening member may be fitted to the guide pipe 1240. The fastening member 1250 may include a hollow 1250a formed inside the fastening member 1250, and a portion of the guide pipe 1240 may be received in the hollow. The surgical tool 1400 may be introduced and discharged through the guide pipe 1240 and a hole 1250b formed in the fastening member 1250. Referring to FIG. 2, the first introduction/discharge port 1200a formed in the steering assembly 1200 may be provided by the hole 1250b formed in the fastening member 1250.

According to one embodiment, the guide pipe 1240 may include fastening grooves 1240a and 1240b formed on an outer peripheral surface of the guide pipe 1240. The fastening member 1250 may include a protrusion part 1250c received in the fastening grooves 1240a and 1240b. The protrusion part 1250c may protrude inward from an inner peripheral surface of the fastening member 1250. The fastening grooves 1240a and 1240b may include a first portion 1240a extending parallel to the first axis A1. The fastening grooves 1240a and 1240b may include a second portion 1240b extending in a circumferential direction of the guide pipe 1240.

Referring to FIGS. 11c to 11e, according to one use example, when the user couples the fastening member 1250 to the guide pipe 1240, first, the fastening member 1250 may be aligned such that the protrusion part 1250c may be inserted into the first portion 1240a, and the fastening member 1250 may be pushed in a direction parallel to the first axis A1. Next, the user may rotate the protrusion part 1250c about the first axis A1 to allow the protrusion part 1250c to move along the second portion 1240b, so that the fastening member 1250 may be finally coupled to the guide pipe 1240.

A first inclined surface 1214b may be formed at an end of the support shaft 1214a. A second inclined surface 1250d having the same inclination as the first inclined surface 1214b may be formed at an end of the fastening member 1250. When the fastening member 1250 is primarily fitted to the guide pipe 1240 along the first portion 1240a, the first inclined surface 1214b and the second inclined surface 1250d may be spaced apart from each other. As the fastening member 1250 secondarily rotates along the second portion 1240b of the guide pipe 1240, the first inclined surface 1214b and the second inclined surface 1250d may become closer to each other. Accordingly, the guide pipe 1240 and the fastening member 1250 may be coupled to each other.

The support shaft 1214a may further include a third inclined surface 1214c that is distinguished from the first inclined surface 1214b and having the same inclination as the first inclined surface 1214b. The fastening member 1250 may further include a fourth inclined surface 1250e having the same inclination as the third inclined surface 1214c. According to the present embodiment, interaction between the third inclined surface 1214c and the fourth inclined surface 1250e may be the same as the interaction between the first inclined surface 1214b and the second inclined surface 1250d.

Referring to FIGS. 11c to 11e, the drap 1260 may also be disposed between the support shaft 1214a and the guide pipe 1240. Accordingly, contaminants may be effectively removed by removing only the drap 1260 from the steering assembly 1200.

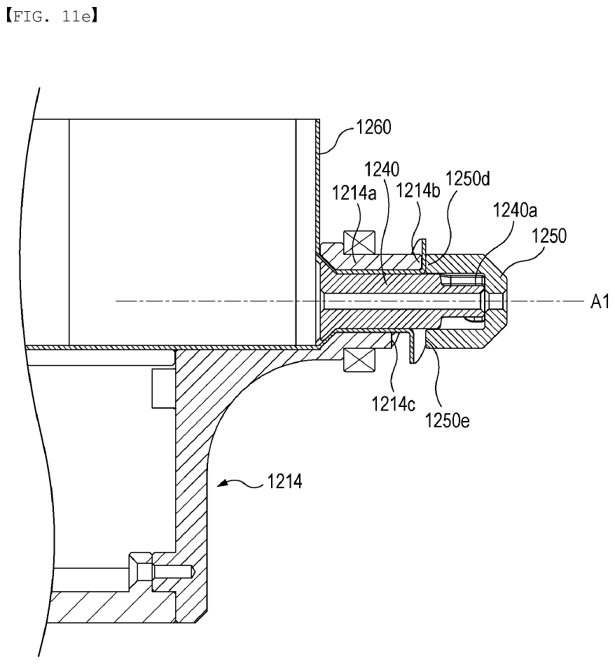

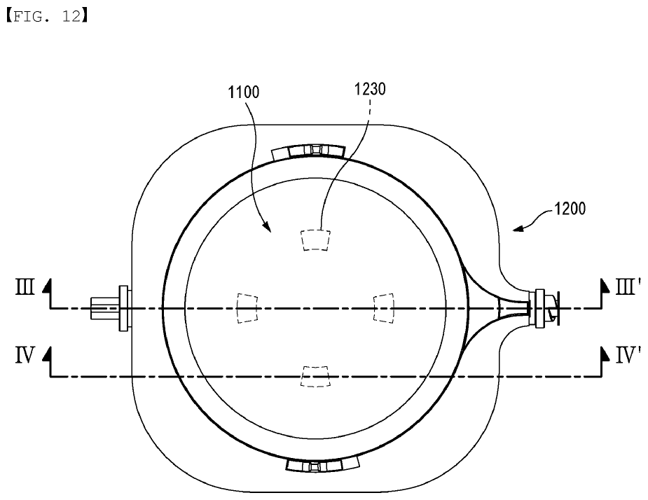

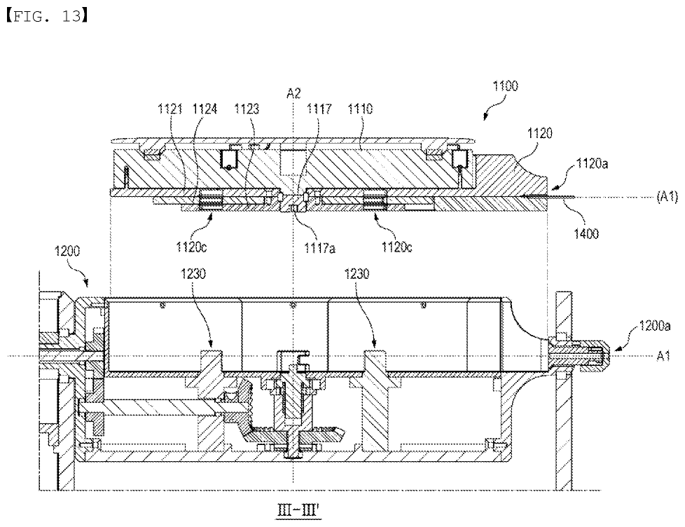

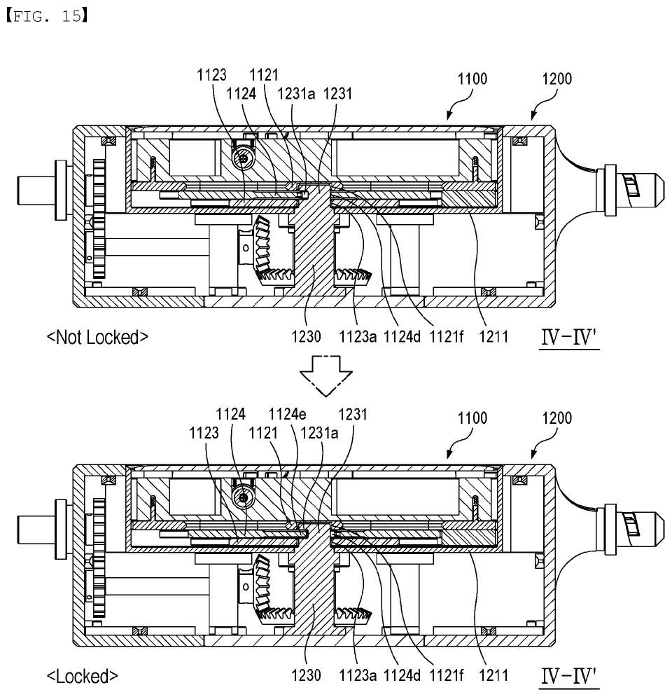

FIG. 12 is a top view showing the steering assembly 1200 on which the drum assembly 1100 is mounted according to one embodiment. FIG. 13 is an exploded sectional view showing the steering assembly 1200 and the drum assembly 1100. FIG. 14 shows an operation of engaging the docking shaft 1222 with the drum assembly 1100. FIG. 15 shows a process of fixing the drum assembly 1100 to the steering assembly 1200.

FIGS. 13 and 14 are sectional views showing the drum assembly 1100 and the steering assembly 1200 of FIG. 12 taken along a line III-III′. FIG. 15 is a sectional view showing the drum assembly 1100 and the steering assembly 1200 of FIG. 12 taken along a line IV-IV′.

Referring to FIGS. 12 and 13, the drum assembly 1100 may include a post reception part 1120c configured to partially receive the post 1230 when the drum assembly 1100 is seated on the steering assembly 1200. Referring to FIG. 15, the post reception part 1120c may be defined by the recess 1121f and the holes 1123a and 1124d, which are formed in the first part 1121, the third part 1123, and the locking member 1124.

Referring to FIG. 13, the steering assembly 1200 may include a first introduction/discharge port 1200a aligned on the first axis A1. The drum assembly 1100 may include a second introduction/discharge port 1120a aligned on the first axis A1. The surgical tool 1400 may be withdrawn out of or inserted into the vascular interventional surgery device 1000 through the first introduction/discharge port 1200a and the second introduction/discharge port 1120a.

The drum assembly 1100 may include a second introduction/discharge port 1120a aligned on the first axis A1. The drum assembly 1100 may be configured to allow the surgical tool 1400 to be inserted into the drum assembly 1100 or withdrawn from the drum assembly 1100 through the second introduction/discharge port 1120a.

Referring to FIG. 14, the connection part 1117 may be engaged with the docking shaft 1222. The connection part 1117 may include a reception part 1117a in which the first protrusion part 1222a of the docking shaft 1222 may be received. For example, the first protrusion part 1222a of the docking shaft 1222 may be provided in a straight shape, and the reception part 1117a of the connection part 1117 may also be provided in a straight shape.

When the drum assembly 1100 is assembled with the steering assembly 1200, the docking shaft 1222 and the connection part 1117 may not be immediately engaged with each other. In this case, as shown in an upper drawing of FIG. 14, the docking shaft 1222 may be pressed by the connection part 1117 to retract to an inner side of the translational interworking shaft 1221. The connection part 1117 may be fixed, and a force pushing upward may be applied to the docking shaft 1222 by the elastic member 1223. In this state, when the vascular interventional surgery device 1000 operates to rotate the translational interworking shaft 1221, at one time point, the docking shaft 1222 may be pushed upward so that the docking shaft 1222 and the connection part 1117 may be engaged with each other (see a lower drawing of FIG. 14). After the connection part 1117 and the docking shaft 1222 are engaged with each other, according to the rotation of the translational interworking shaft 1221 about the second axis A2, the rotation unit 1110 may rotate about the second axis A2 with respect to the steering assembly 1200 (or the guide unit 1120).

Referring to FIGS. 13 and 15, the post 1230 may include a protrusion part 1231 protruding upward from the upper housing 1211. The protrusion part 1231 may include a locking recess 1231a selectively engaged with a portion of the locking member 1124.

According to the present embodiment, while the drum assembly 1100 is not fixed to the steering assembly 1200 (see an upper drawing of FIG. 15), the recess 1121f of the first part 1121, the hole 1123a of the third part 1123, and the hole 1124d of the locking member 1124 may all be aligned with each other. In this state, the locking member 1124 may not be located in the locking recess 1231a formed in the protrusion part 1231 of the post 1230. In this state, the drum assembly 1100 may be freely separated from the steering assembly 1200. Referring to FIG. 6 and a lower drawing of FIG. 15, as the locking member 1124 rotates about the second axis A2, a portion of the locking member 1124 may be located in the locking recess 1231a formed in the protrusion part 1231 of the post 1230. As the portion of the locking member 1124 is inserted into the locking recess 1231a, the drum assembly 1100 may be fixed to the steering assembly 1200.

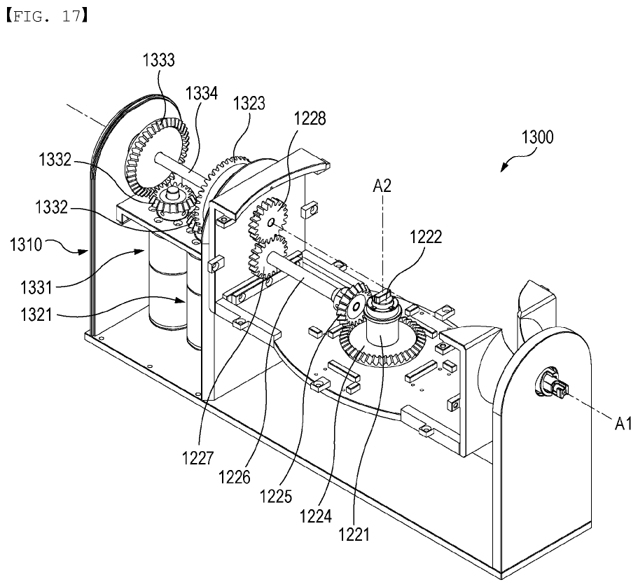

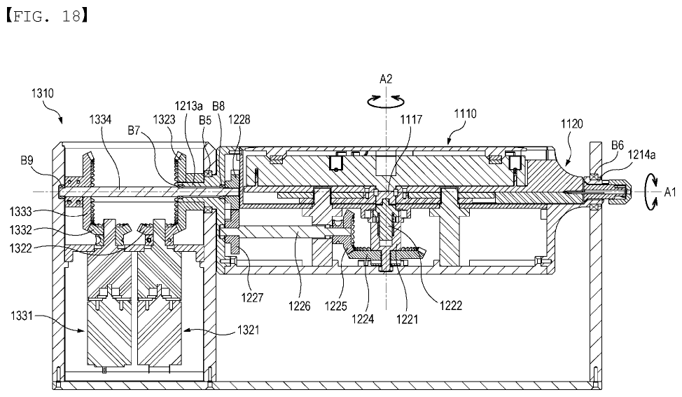

FIG. 16 is an exploded perspective view showing a base 1300 according to one embodiment. FIG. 17 shows a driving unit of the vascular interventional surgery device 1000 according to one embodiment. FIG. 18 is a sectional view showing the driving unit of the vascular interventional surgery device 1000.

The base 1300 may include a fixed frame 1310. The fixed frame 1310 may include a lower plate 1311, support frames 1312, 1313, and 1314, and an upper cover 1315. Referring to FIGS. 16 to 18, a first support frame 1312 and a second support frame 1313 may be configured to support the steering driving shaft 1213a and the support shaft 1214a of the steering assembly 1200.

Some driving elements configured to rotate the steering assembly 1200 or the rotation unit 1110 may be mounted on the fixed frame 1310. For example, the translational actuator 1331 and the steering actuator 1321 may be installed on a plate 1316 installed between the second support frame 1313 and a third support frame 1314. As another example, the translational driving shaft 1334 may be partially supported by the third support frame 1314.

The vascular interventional surgery device 1000 may include a steering driving unit configured to rotate the steering assembly 1200 about the first axis A1. The steering driving unit may include a steering driving shaft 1213a extending along the first axis A1 on one side of the steering assembly 1200 and rotatably coupled to the fixed frame 1310, and a steering actuator 1321 mechanically connected to the steering driving shaft 1213a.

The vascular interventional surgery device 1000 may include a translational driving unit configured to rotate the rotation unit 1110 about the second axis A2 with respect to the steering assembly 1200.

The translational driving unit may include a translational driving shaft 1334 extending along the first axis A1. The translational driving unit may include a translational interworking shaft 1221 extending along the second axis A2 and configured to mechanically interwork with the translational driving shaft 1334. The translational driving unit may include a translational actuator 1331 mechanically connected to the translational driving shaft 1334.

Both the steering driving shaft 1213a and the translational driving shaft 1334 may extend along the first axis A1. The steering driving shaft 1213a and the translational driving shaft 1334 may form a dual-axis structure. For example, the steering driving shaft 1213a may include a hollow formed inside the steering driving shaft 1213a and through which the translational driving shaft 1334 may pass. The translational driving shaft 1334 may rotate independently of the rotation of the steering assembly 1200. The translational driving shaft 1334 may transmit a driving force to rotate the rotation unit 1110 about the second axis A2.

One side of the translational driving shaft 1334 may be supported by the third support frame 1314. An opposite side of the translational driving shaft 1334 may be supported by the steering driving shaft 1213a.

The translational driving unit may include a translational interworking shaft 1221 extending along the second axis A2. The translational driving unit may include a translational actuator 1331 mechanically connected to the translational interworking shaft 1221. The translational driving unit may include a docking shaft 1222 coupled to the translational interworking shaft 1221 so as to be retractable along the second axis A2.

The translational driving unit may include a translational actuator 1331. The translational driving unit may include a first bevel gear 1332 coupled to the translational actuator 1331. A second bevel gear 1333 engaged with the first bevel gear 1332 may be coupled to one end of the translational driving shaft 1334. A first spur gear 1228 may be coupled to an opposite end of the translational driving shaft 1334. The translational driving unit may include an intermediate shaft 1226 having one end to which a second spur gear 1227 engaged with the first spur gear 1228 is coupled. A third bevel gear 1225 may be coupled to an opposite end of the intermediate shaft 1226. The translational interworking shaft 1221 may be coupled to a fourth bevel gear 1224 engaged with the third bevel gear 1225. The translational driving unit may include a docking shaft 1222 retractably coupled to the translational interworking shaft 1221.

The steering driving unit may include a steering actuator 1321. The steering driving unit may include a fifth bevel gear 1322 coupled to the steering actuator 1321. A sixth bevel gear 1323 engaged with the fifth bevel gear 1322 may be coupled to the steering driving shaft 1213a.

Referring to FIGS. 8, 9, and 16 to 18, according to one embodiment, bearings (e.g., B1, B2, B3, B4, B5, B6, B7, B8, and B9) may be mounted on the shafts constituting the steering driving unit or the translational driving unit (e.g., the steering driving shaft 1213a, the translational driving shaft 1334, the translational interworking shaft 1221, and the intermediate shaft 1226) to reduce rotational friction.

Although the technical idea of the present disclosure has been described above by some embodiments and examples shown in the accompanying drawings, it should be noted that various substitutions, modifications, and changes can be made without departing from the technical idea and scope of the present disclosure, which may be understood by a person having ordinary skill in the art to which the present disclosure pertains. In addition, such substitutions, modifications, and changes should be construed to fall within the scope of the appended claims.

1. A vascular interventional surgery device comprising:
a fixed frame;
a steering assembly coupled to the fixed frame so as to be rotatable a first axis, about and including a first introduction/discharge port aligned on the first axis; and
a drum assembly coupled to the steering assembly so as to be rotatable about a second axis extending in a direction that is transverse to the first axis, including a rotation unit configured to allow a flexible wire-type or tubular surgical tool that is insertable into a vessel to be wound in a circumferential direction about the second axis, and configured to guide the surgical tool such that the surgical tool is withdrawn outward or inserted inward through the first introduction/discharge port when the rotation unit rotates about the second axis.

2. The vascular interventional surgery device of claim 1, wherein the drum assembly includes a second introduction/discharge port aligned on the first axis, and is configured to allow the surgical tool to be inserted into the drum assembly or withdrawn from the drum assembly through the second introduction/discharge port.

3. The vascular interventional surgery device of claim 1, wherein the drum assembly includes a guide unit coupled to the steering assembly, and
the rotation unit is rotatably coupled to the guide unit.

4. The vascular interventional surgery device of claim 3, wherein the rotation unit includes a reception part extending in the circumferential direction and configured to allow at least a portion of the surgical tool to be wound and received.

5. The vascular interventional surgery device of claim 4, wherein the guide unit includes a second introduction/discharge port aligned on the first axis, and a passage configured to guide the surgical tool between the second introduction/discharge port and the reception part.

6. The vascular interventional surgery device of claim 5, wherein the guide unit includes a rib inserted into the reception part and defining at least a portion of the passage.

7. The vascular interventional surgery device of claim 6, wherein the portion of the passage is defined by an inclined surface formed on the rib.

8. The vascular interventional surgery device of claim 6, wherein the portion of the passage formed by the rib extends in a direction between a direction that is parallel to the second axis and the circumferential direction.

9. The vascular interventional surgery device of claim 1, further comprising:
a steering driving unit configured to rotate the steering assembly about the first axis with respect to the fixed frame,
wherein the steering driving unit includes:
a steering driving shaft extending along the first axis from one side of the steering assembly and rotatably coupled to the fixed frame; and
a steering actuator mechanically connected to the steering driving shaft.

10. The vascular interventional surgery device of claim 9, further comprising:
a translational driving unit configured to rotate the rotation unit about the second axis with respect to the steering assembly,
wherein the translational driving unit includes:
a translational driving shaft extending along the first axis and passing through an inside of the steering driving shaft;
a translational interworking shaft extending along the second axis and configured to mechanically interwork with the translational driving shaft; and
a translational actuator mechanically connected to the translational driving shaft.

11. The vascular interventional surgery device of claim 1, further comprising:
a translational driving unit configured to rotate the rotation unit about the second axis with respect to the steering assembly,
wherein the translational driving unit includes:
a translational interworking shaft extending along the second axis;
a translational actuator mechanically connected to the translational interworking shaft; and
a docking shaft coupled to the translational interworking shaft so as to be retractable along the second axis, and
the rotation unit includes a docking groove selectively engaged with the docking shaft.

12. The vascular interventional surgery device of claim 11, wherein the translational interworking shaft includes a hollow formed inside the translational interworking shaft and configured to receive the docking shaft, and
the translational driving unit includes an elastic member interposed between the translational interworking shaft and the docking shaft.

13. The vascular interventional surgery device of claim 1, wherein the drum assembly is detachably coupled to the steering assembly.

14. The vascular interventional surgery device of claim 13, wherein the steering assembly includes a post, and
the drum assembly includes a locking member selectively engaged with the post.

15. The vascular interventional surgery device of claim 14, wherein the post is selectively engaged with the locking member according to rotation of the locking member.

16. The vascular interventional surgery device of claim 15, wherein the locking member includes a disk selectively engaged with the post, an arm extending radially from the disk, and a lever extending from the arm in a direction that is parallel to a rotation axis of the locking member.

17. A drum assembly for a vascular interventional surgery device, the drum assembly comprising:
a guide unit including an introduction/discharge port aligned on a first axis; and
a rotation unit coupled to the guide unit so as to be rotatable about a second axis extending in a direction that is transverse to the first axis, and configured to allow a flexible wire-type or tubular surgical tool that is insertable into a vessel to be wound in a circumferential direction about the second axis,
wherein the guide unit is configured to guide the surgical tool such that the surgical tool advances outward or retracts inward through the introduction/discharge port when the rotation unit rotates about the second axis with respect to the guide unit.

18. The drum assembly of claim 17, wherein the rotation unit includes a reception part extending in the circumferential direction and configured to allow at least a portion of the surgical tool to be wound and received.

19. The drum assembly of claim 18, wherein the guide unit includes a passage configured to guide the surgical tool between the introduction/discharge port and the reception part, and includes a rib inserted into the reception part and defining at least a portion of the passage.

20. The drum assembly of claim 19, wherein the portion of the passage is defined by an inclined surface formed obliquely on the rib.
